(19) United States
(12) Patent Application Publication
(10) Pub. No.: US 2016/0206288 A1
(43) Pub. Date: Jul. 21, 2016

(54) ULTRASONIC PROBE, ULTRASONIC IMAGING APPARATUS, AND METHOD FOR CONTROLLING THE SAME

(71) Applicant: SAMSUNG ELECTRONICS CO., LTD., Suwon-si (KR)

(72) Inventors: Minseog CHOI, Seoul (KR); Youngil KIM, Suwon-si (KR); Jong Keun SONG, Yongin-si (KR); Eunsung LEE, Hwaseong-si (KR)

(73) Assignee: SAMSUNG ELECTRONICS CO., LTD., Suwon-si (KR)

(21) Appl. No.: 14/868,606

(22) Filed: Sep. 29, 2015

(30) Foreign Application Priority Data
Jan. 20, 2015 (KR) 10-2015-0009235

(51) Int. Cl. A61B 8/08; A61B 8/00

(52) U.S. Cl. CPC A61B 8/5207; A61B 8/56; A61B 8/5215; A61B 8/4444

(57) ABSTRACT

An ultrasonic probe includes an optical resonating waveguide configured to receive an echo ultrasound signal; a calculator configured to calculate an acoustic pressure of the echo ultrasound signal, based on a change in wavelength of an optical signal traveling within the optical resonating waveguide, the change occurring in response to the optical resonating waveguide receiving the echo ultrasound signal; and a converter configured to convert the echo ultrasound signal to an electric signal based on the acoustic pressure of the echo ultrasound signal.

### CROSS-REFERENCE TO RELATED APPLICATION(S)

This application claims priority under 35 U.S.C. §119(a) to Korean patent application No. 10-2015-0009235, filed on Jan. 20, 2015 in the Korean Intellectual Property Office, the entire disclosure of which is incorporated hereby incorporated by reference in its entirety.

### BACKGROUND

1. Field

The exemplary embodiments consistent with the present disclosure relate to an ultrasonic probe, ultrasonic imaging apparatus, and method for controlling the same, which performs conversion between ultrasound and electric signals.

2. Description of the Related Art

Transducers of an ultrasonic probe use piezoelectric materials for generating acoustic waves in proportion to an electric field applied thereto, and conversely generating electric signals in proportion to an external acoustic pressure. Membrane-type transducers have recently been introduced, such as capacitive Micromachined Ultrasonic Transducers (cMUT), piezoelectric Micromachined Ultrasonic Transducers, etc., which have wide bandwidth characteristics, in order to increase the resolution of an ultrasound image.

These Membrane-type transducers may be applied not only to televisions but also to display devices used in various fields, and thus, the forms of the display devices are becoming more diverse.

### SUMMARY

In accordance with an aspect of an exemplary embodiment, an ultrasonic probe is provided. The ultrasonic probe includes an optical resonating waveguide configured to receive an echo ultrasound signal; a calculator configured to calculate an acoustic pressure of the echo ultrasound signal, based on a change in wavelength of an optical signal traveling within the optical resonating waveguide, the change occurring in response to the optical resonating waveguide receiving the echo ultrasound signal; and a converter configured to convert the echo ultrasound signal to an electric signal based on the acoustic pressure of the echo ultrasound signal.

The optical resonating waveguide may include a linear waveguide and a circular waveguide, and the calculator may be configured to detect the change in the wavelength of the optical signal being resonated and traveling from the linear waveguide to the circular waveguide based on at least one of a change in refractive index and physical deformation of the optical resonating waveguide that occur in response to the optical resonating waveguide receiving the echo ultrasound signal.

The optical resonating waveguide may include a linear waveguide and a circular waveguide, and the converter may be configured to determine a change in intensity of the optical signal traveling from the linear waveguide to the circular waveguide based on the acoustic pressure of the echo ultrasound signal, and convert the echo ultrasound signal to the electric signal based on the acoustic pressure of the echo ultrasound signal and the change in the intensity of the optical signal.

The ultrasonic probe may further include a controller configured to control a flow of the optical signal within the optical resonating waveguide.

The ultrasonic probe may further include at least one of a piezoelectric layer and a piezoelectric membrane, and a transmitter configured to transmit an ultrasound signal converted from an electric signal based on at least one of the piezoelectric layer and the piezoelectric membrane to a subject.

The optical resonating waveguide may further include a linear waveguide and a circular waveguide, and each of the linear waveguide and the circular waveguide including a core and a cladding, and the core and the cladding may be implemented as at least one of a circular shape and a polygonal shape.

The cladding may be implemented as a lens configured to focus an ultrasound signal to be transmitted to a subject.

In accordance with another aspect of an exemplary embodiment, an ultrasonic imaging apparatus includes an optical resonating waveguide configured to receive an echo ultrasound signal; a converter configured to output an electric signal according to an acoustic pressure of the ultrasound signal; a signal processor configured to obtain ultrasound image data based on the electric signal; and an image processor configured to generate an ultrasound image based on the ultrasound image data.

The optical resonating waveguide may further include a linear waveguide and a circular waveguide, and the ultrasound imaging apparatus may further include a calculator configured to detect a change in wavelength of an optical signal being resonated and traveling from the linear waveguide to the circular waveguide based on at least one of a change in refractive index and physical deformation of the optical resonating waveguide that occur in response to the optical resonating waveguide receiving the echo ultrasound signal, and calculate the acoustic pressure of the echo ultrasound signal based on the change in the wavelength.

The optical resonating waveguide may include a linear waveguide and a circular waveguide, and the converter may be configured to determine a change in intensity of an optical signal traveling from the linear waveguide to the circular waveguide based on the acoustic pressure of the echo ultrasound signal, and output an electric signal converted based on the acoustic pressure of the echo ultrasound signal and the change in the intensity of the optical signal.

The ultrasound imaging apparatus may further include at least one of a piezoelectric layer and a piezoelectric membrane, and a transmitter configured to transmit an ultrasound signal converted from an electric signal based on at least one of the piezoelectric layer and the piezoelectric membrane to a subject.

The optical resonating waveguide may further include a linear waveguide and a circular waveguide, and each of the linear waveguide and the circular waveguide may include a core and a cladding, and the core and the cladding may be implemented as at least one of a circular shape and a polygonal shape.

The cladding may be implemented as a lens configured to focus an ultrasound signal to be transmitted to a subject.

In accordance with another aspect of an exemplary embodiment, a method for controlling an ultrasonic imaging apparatus includes receiving, by an optical resonating waveguide, an echo ultrasound signal; outputting an electric signal according to an acoustic pressure of the echo ultrasound signal; obtaining ultrasound image data based on the electric signal; and generating an ultrasound image based on the ultrasound image data.

The optical resonating waveguide may include a linear waveguide and a circular waveguide, and the outputting may include detecting a change in a wavelength of an optical signal being resonated and traveling from the linear waveguide to the circular waveguide based on at least one of a change in refractive index and physical deformation of the optical resonating waveguide that occur upon the receiving of the echo ultrasound signal.

The optical resonating waveguide may include a linear waveguide and a circular waveguide, and the outputting may include determining a change in intensity of an optical signal traveling from the linear waveguide to the circular waveguide based on the acoustic pressure of the echo ultrasound signal, and outputting the electric signal which is converted based on the acoustic pressure of the echo ultrasound signal and the change in the intensity of the optical signal.

The ultrasound imaging apparatus may include at least one of a piezoelectric layer and a piezoelectric membrane, and the method may further include transmitting an ultrasound signal converted from an electric signal based on the at least one of the piezoelectric layer and the piezoelectric membrane to a subject.

The optical resonating waveguide may further include a linear waveguide and a circular waveguide, and each of the linear waveguide and the circular waveguide may include a core and a cladding, and the core and the cladding may be implemented as at least one of a circular shape and a polygonal shape.

The cladding may be implemented as a lens configured to focus an ultrasound signal to be transmitted to a subject.

Other aspects, advantages, and salient features of the exemplary embodiments will become apparent to those skilled in the art from the following detailed description, which, taken in conjunction with the annexed drawings, discloses exemplary embodiments of the disclosure.

### BRIEF DESCRIPTION OF THE DRAWINGS

The above and other features and advantages of the exemplary embodiments will become more apparent by describing in detail exemplary embodiments thereof with reference to the attached drawings in which:

FIG. 1 illustrates exterior components of an ultrasonic imaging apparatus, according to an exemplary embodiment;

FIG. 2 illustrates an exterior view of an ultrasonic probe that includes one dimensional (1D) array transducers, according to an exemplary embodiment;

FIG. 3 illustrates an exterior view of an ultrasonic probe that includes two dimensional (2D) array transducers, according to an exemplary embodiment;

FIG. 4 is a block diagram of an ultrasonic imaging apparatus including an ultrasonic probe and a main unit, according to an exemplary embodiment;

FIG. 5 illustrates an internal structure of an ultrasonic probe in a piezoelectric Micromachined Ultrasound Transducer (pMUT) type, according to an exemplary embodiment;

FIG. 6 illustrates an internal structure of an ultrasonic probe in a piezoelectric ultrasonic transducer type, according to an exemplary embodiment;

FIG. 7 illustrates a spectrum of an optical signal and optical delay signal, according to an exemplary embodiment;

FIG. 8 illustrates structures of a core and a cladding, according to an exemplary embodiment; and

FIG. 9 is a flowchart illustrating a method for controlling an ultrasonic imaging apparatus, according to an exemplary embodiment.

Throughout the drawings, like reference numerals will be understood to refer to like parts, components, and structures.

### DETAILED DESCRIPTION

Exemplary embodiments of the present disclosure will be described in detail with reference to accompanying drawings.

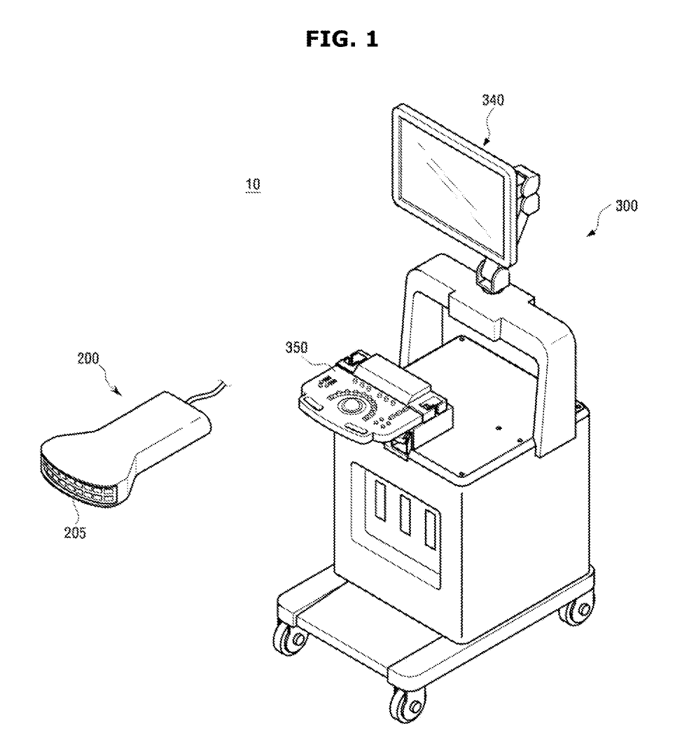

FIG. 1 illustrates exterior components of an ultrasonic imaging apparatus, according to an exemplary embodiment.

Referring to FIG. 1, an ultrasonic imaging apparatus 10 includes an ultrasonic probe 200 for transmitting ultrasound signals to a subject and converting echo ultrasound signals received from the subject to electric signals, and a main unit 300 for creating an ultrasound image based on the ultrasound signal. The main unit 300 may be connected to the ultrasonic probe 200 over a wired or wireless communication network. The main unit 300 may correspond to a workstation equipped with a display 340 and an input unit 350.

According to an exemplary embodiment, the ultrasonic probe 200 may be connected to the main unit 300 over a wireless communication network for receiving various signals required to control the ultrasonic probe 200, or for delivering an analog or digital signal corresponding to an echo ultrasound signal received by the ultrasonic probe 200.

According to an exemplary embodiment, the wireless communication network refers to a communication network for wireless signal communication. The main body 300 may perform wireless communication with the ultrasonic probe 200 through at least one of a short-range communication module and a mobile communication module.

According to an exemplary embodiment, the short-range communication module refers to a module for near distance communication within a predetermined range. For example, the short-range (e.g., near distance) communication may include Wireless Local Area Network (WLAN), Wi-Fi, Bluetooth, Zigbee, Wi-Fi Direct (WFD), Ultra Wideband (UWB), Infrared Data Association (IrDA), Bluetooth Low Energy (BLE), Near Field Communication (NFC), etc., but is not limited thereto.

The mobile communication module may transmit and receive Radio Frequency (RF) signals to and from one of the base stations, external terminals, and servers in the mobile communication network. The RF signal refers to a signal including various types of data. The main unit 300 may exchange signals including various types of data with the ultrasonic probe 200 via at least one of the base stations and servers.

For example, the main unit 300 may exchange signals including various types of data with the ultrasonic probe 200 via a base station in a mobile communication network, e.g., a 3G or 4G mobile communication network. In addition, the main unit 300 may exchange data with a hospital server or another type of medical equipment in the hospital through the Picture Archiving and Communication System (PACS). Furthermore, the main unit 300 may exchange data according to the Digital Imaging and Communications in Medicine (DICOM) standard, although is not limited thereto.

Moreover, the main unit 300 may exchange data with the ultrasonic probe 200 over a wired communication network. The wired communication network refers to a communication network for wired signal communication. According to an exemplary embodiment, the main unit 300 may exchange various signals with the ultrasonic probe 200 using the wired communication network according to various protocols, such as Peripheral Component Interconnect (PCI), PCI-express, Universe Serial Bus (USB), etc., although is not limited thereto.

The main unit 300 of the ultrasonic imaging apparatus 10 may include the display 340 and the input unit 350 (e.g., inputter). The input unit 350 may receive setting information for the ultrasonic probe 200, and may receive various control commands from the user.

According to an exemplary embodiment, the setting information for the ultrasonic probe 200 may include information about gain, zoom, focus, Time Gain Compensation (TGC), depth, frequency, power, frame average, dynamic range, etc. The setting information for the ultrasonic probe 200, however, is not limited thereto, and may include other types of information that may be set to capture ultrasound images.

The information may be delivered to the ultrasonic probe 200 over the wired or wireless communication network, and the ultrasonic probe 200 may then be configured according to the information. Moreover, the main unit 300 may receive various control commands, such as a command to transmit an ultrasound signal, from the user through the input unit 350, and forward the command to the ultrasonic probe 200.

The input unit 350 may be implemented as a keyboard, a foot switch, or a foot pedal. The keyboard may, for example, be implemented as hardware. The keyboard may include at least one of switches, keys, a joy stick, and a trackball. Alternatively, the keyboard may be implemented as software, such as a graphic user interface. In this case, the keyboard may be displayed through the main display 340. The foot switch or foot pedal may be placed in a lower part of the main unit 300, and the user may use the foot pedal to control operation of the ultrasonic imaging apparatus 10.

The display 340 may be implemented using various well-known display technologies, such as, for example, Cathode Ray Tube (CRT), Liquid Crystal Display (LCD), Light Emitting Diodes (LEDs), Plasma Display Panel (PDP), Organic Light Emitting Diodes (OLEDs), etc., but is not limited thereto.

The display 340 may display an ultrasound image of a target part inside the subject. The ultrasound image displayed on the display 340 may be a two dimensional (2D) or three dimensional (3D) ultrasound image, and various ultrasound images may be displayed depending on operating modes of the ultrasonic imaging apparatus 10. The display 340 may also display menus or user guides for ultrasonic diagnosis, and information regarding operating status of the ultrasonic probe 200.

According to an exemplary embodiment, the ultrasound image may include an Amplitude mode (A-mode) image, Brightness Mode (B-mode) image, and Motion mode (M-mode) image, and may further include a Color mode (C-mode) image and Doppler mode (D-mode) image.

An A-mode image refers to an ultrasound image representing an amplitude of an ultrasound signal corresponding to an echo ultrasound signal. A B-mode image refers to an ultrasound image representing a magnitude of an ultrasound signal corresponding to an echo ultrasound signal by a brightness level. An M-mode image refers to an ultrasound image representing movements of the subject over time at a particular position. A D-mode image refers to an ultrasound image representing a moving subject in waveforms based on the Doppler effect, and a C-mode image refers to an ultrasound image representing a moving subject in a color spectrum.

In case the display 340 is implemented as a type of touch screen, the display 340 may serve as the input unit 350 as well. The main unit 300 may receive various commands from the user through at least one of the display 340 and the input unit 350.

Additionally, the main unit 300 may include a voice recognition sensor to receive a voice command from the user. The ultrasonic probe will now be described in more detail.

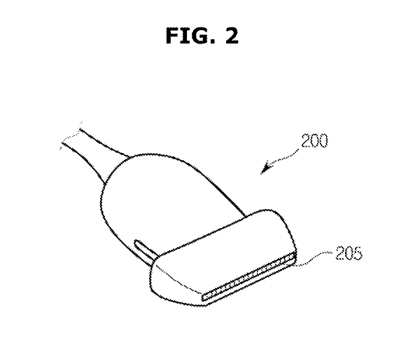

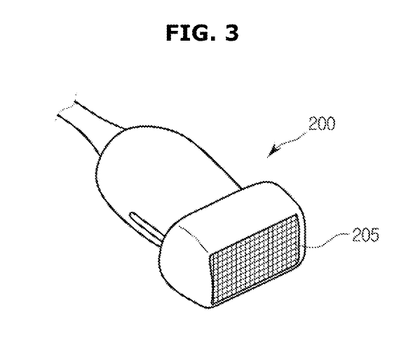

FIG. 2 illustrates an exterior view of an ultrasonic probe that includes one dimensional (1D) array transducers, according to an exemplary embodiment, and FIG. 3 illustrates an exterior view of an ultrasonic probe that includes 2D array transducers, according to an exemplary embodiment.

The ultrasonic probe 200 is a device that makes contact with the surface of a subject, and is configured to transmit and/or receive ultrasound signals. Specifically, the ultrasonic probe 200 may serve to transmit an ultrasound signal to a particular part inside the subject in response to a signal received from the main unit 300, and receive an echo ultrasound signal reflected from the particular part inside the subject and deliver the received echo ultrasound signal to the main unit 300. The echo ultrasound signal may be, but is not limited to being, an ultrasound signal, which is an RF signal reflected from the subject, or may be any signal resulting from reflection of the ultrasound signal transmitted to the subject.

According to an exemplary embodiment, the term “subject”, as herein used, may refer to a living body of a human or animal, although is not limited thereto, and may be any object whose internal structure may be imaged according to the ultrasound signal.

The ultrasonic probe 200 may include a transducer array for performing conversion between electric and ultrasound signals to transmit an ultrasound signal into the subject or receive an echo ultrasound signal from the subject. The transducer array may include a plurality of transducer elements.

The ultrasonic probe 200 may generate ultrasound signals through the transducer array, focus and transmit the ultrasound signals to a target part inside the subject, and receive echo ultrasound signals reflected from the target part inside the subject through the transducer array.

When the echo ultrasound signal reaches the transducer array, the transducer array may vibrate at a certain frequency that corresponds to a frequency of the echo ultrasound signal, and output an alternate current at a frequency corresponding to the vibration frequency of the transducer array. Accordingly, the transducer array may be able to convert the received echo ultrasound signal to an electric echo signal.

The transducer array may be a 1D array or a 2D array. According to an exemplary embodiment, the transducer module 205 may include a 1D transducer array as shown in FIG. 1.

The transducer elements of the 1D transducer array may each perform conversion between ultrasound and electric signals. To achieve this feature, the transducer elements may be implemented with magnetostrictive ultrasonic transducers that use magnetostrictive effects of a magnetic substance, piezoelectric ultrasonic transducers that use piezoelectric effects of a material, piezoelectric micromachined ultrasonic transducers (pMUTs), or the like, or may also be implemented with capacitive micromachined ultrasonic transducers (cMUTs) that transmit or receive ultrasounds using vibration of hundreds or thousands of micromachined thin films.

The ultrasonic probe 200 may include the transducer module 205 configured in a linear array, as shown in FIG. 2, or configured in a convex array. The basic operating principle of the ultrasonic probe 200 is equally applied to both cases, but in case of the ultrasonic probe 200 having the transducer module 205 in the convex array, ultrasound signals irradiated from the transducer module 205 are fan-shaped and thus the resultant ultrasound image may also be fan-shaped.

According to another example, the transducer module 205 may include a 2D transducer array, as shown in FIG. 3. With the 2D transducer array, the internal part of the subject may be imaged in three dimensions. Furthermore, even if the transducer array of the ultrasonic probe 200 is a 1D array, the ultrasonic probe 200 may obtain volume information of the inside of the subject while mechanically moving the 1D transducer array, thus delivering an echo ultrasound signal to the main unit 300, based on which a 3D ultrasound image may be created.

Each of the transducer elements included in the 2D transducer array may be the same as that of the 1D transducer array. The ultrasonic probe and ultrasonic imaging apparatus including the ultrasonic probe will now be described in more detail.

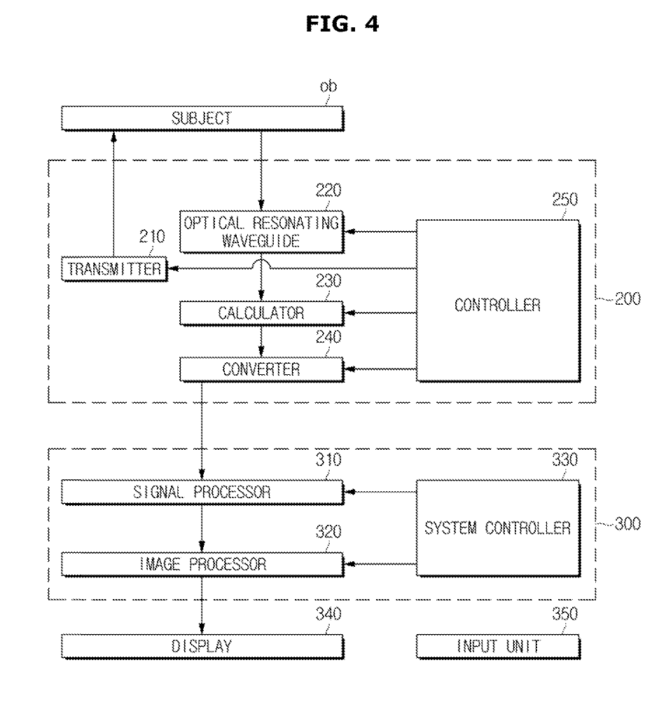

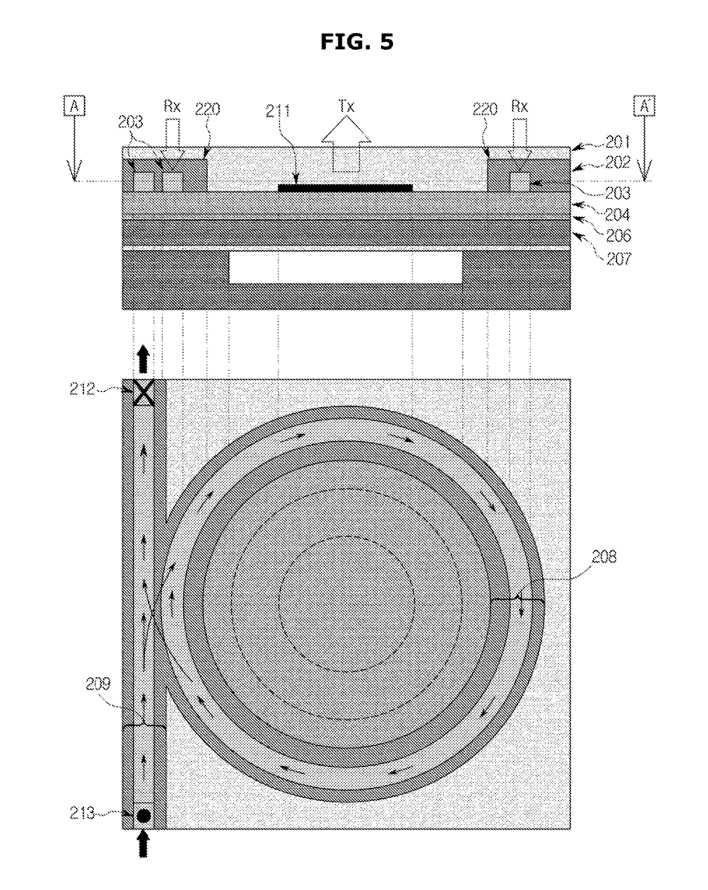

FIG. 4 is a block diagram of an ultrasonic imaging apparatus including an ultrasonic probe and a main unit, according to an exemplary embodiment. In the following description, the internal structure of an ultrasonic probe implemented as a pMUT type as shown in FIG. 5 will also be described.

Referring to FIG. 4, the ultrasonic probe 200 may include a transmitter 210, an optical resonating waveguide 220, a calculator 230, a converter 240, and a controller 250. The calculator 230, the converter 240, and the controller 250 may be integrated in a System On Chip (SOC) embedded in the ultrasonic probe 200. In this regard, there may be multiple SOCs embedded in the ultrasonic probe 200, and the aforementioned components may not be limited to being only integrated in a single SOC.

The transmitter 210 may transmit an ultrasound signal to a subject (ob) in response to a control command from the user. The transmitter 210 may use the transducer elements implemented in various types to convert an electric signal to an ultrasound signal and may transmit the ultrasound signal to the subject.

According to an exemplary embodiment, in case the transducer element is implemented with a piezoelectric ultrasonic transducer element, the transmitter 210 may convert the electric signal to an ultrasound signal through a piezoelectric layer. Specifically, the piezoelectric layer may include a piezoelectric substance, the upper and lower ends of which are connected to electrodes to convert an ultrasound signal to an electric signal or an electric signal to an ultrasound signal. The internal structure of the ultrasonic probe 200 implemented with the piezoelectric ultrasonic transducer elements will be described later.

According to another example, in case the transducer element is implemented with the pMUT element, the transmitter 210 may convert the electric signal to an ultrasound signal through a piezoelectric membrane, which is thinner than the piezoelectric layer, and transmit the ultrasound signal to the subject.

FIG. 5 illustrates an internal structure of an ultrasonic probe in a pMUT type, according to an exemplary embodiment. Referring to FIG. 5, the ultrasonic probe 200 may have an upper electrode 211 and a lower electrode 206. As a voltage is applied across the upper electrode 211 and the lower electrode 206, an electric field is generated, causing a piezoelectric membrane 204 to expand or contract in the left and right direction, which makes the piezoelectric membrane 204 bend to generate an ultrasound signal. In this regard, a structure film 207 may be applied below the piezoelectric membrane 204 for acting to increase bending of the piezoelectric membrane 204 through expansion or contraction of the piezoelectric membrane 204 in the left and right direction. For example, the piezoelectric membrane 204 and the structure film may be implemented as bimetal.

While the conventional ultrasonic probe may receive an echo ultrasound signal reflected from the subject and output an electric signal based on the acoustic pressure of the received echo ultrasound signal through transducer elements, the ultrasonic probe 200 in accordance with an exemplary embodiment may receive an echo ultrasound signal reflected from the subject (ob) through an optical resonating waveguide 220. The acoustic pressure refers to a pressure generated by acoustic waves. The optical resonating waveguide 220 will now be described.

Referring to FIG. 5, the optical resonating waveguide 220 may include a linear waveguide 209 and a circular waveguide 208. As shown in FIG. 5, the linear waveguide 209 may be implemented as a waveguide in a straight line type, and the circular waveguide 208 may be implemented as a waveguide in a circular type. According to an exemplary embodiment, a waveguide refers to a conduit made for the purpose of transmission of a particular signal.

According to an exemplary embodiment, the linear waveguide 209 and circular waveguide 208 may be implemented such that an optical signal flows through the inside of the linear waveguide 209 and circular waveguide 208. Accordingly, the inside of the linear waveguide 209 and circular waveguide 208 may be implemented with a core 203 formed of an optical fiber and a cladding 202 that surrounds the core 203. The term optical fiber as herein used may refer to a fiber configured for the purpose of transmission of optical signals.

In order for an optical signal flowing inside the core 203 not to leak out, e.g., not to propagate into the cladding 202, total reflection of the optical signal should occur within the core 203. According to an exemplary embodiment, total reflection as used herein is a phenomenon in which an optical signal striking a boundary of the core 203 is reflected completely within the core 203 when the optical signal proceeds from a medium having a higher refractive index to a medium having a lower refractive index, because the incident angle is larger than a critical angle. Accordingly, to enable total reflection of an optical signal, the core 203 should be formed of a substance having a refractive index greater than the cladding 202. The optical signal may then be focused in the core 203, proceeding within the core 203 without leaking out into the cladding 202.

More specifically, the lower diagram of FIG. 5 is a sectional view of the optical resonating waveguide 220 cut along the line of A to A′. As shown in the lower diagram of FIG. 5, the optical resonating waveguide 220 may include the linear waveguide 209 and the circular waveguide 208, and the inside of the linear waveguide 208 and circular waveguide 209 may be comprised of the core 203 and the cladding 202. According to an exemplary embodiment, the linear waveguide 209 and circular waveguide 208 may each be implemented as a ring resonator type.

The core 203 and cladding 202 may be implemented as polymer, glass, plastic, or any well-known material. The core 203 may have a rectangular shape, as shown in FIG. 5, but is not limited thereto, and may have, but is not limited to having, at least one of a circular shape and a polygonal shape. The cladding 202 may also have many different shapes.

The linear waveguide 209 may have an input end 213 and an output end 212 for inputting and outputting optical signals, respectively. Accordingly, an optical signal is input through the input end 213 and output through the output end 212. The ultrasonic probe 200 may control input and output of the optical signal with control logic. Positions of the input end 213 and the output end 212 are not limited to the exemplary configuration shown in FIG. 5, and may instead be switched.

As shown in FIG. 5, the circular waveguide 208 has a particular part located close to the linear waveguide 209. Accordingly, among optical signals that proceed within the linear waveguide 209, an optical signal with a resonance frequency may be resonated and propagate into the circular waveguide 208. The resonance frequency may be pre-determined by a developer in a design stage of the optical resonating waveguide 220.

According to an exemplary embodiment, the circular waveguide 208 may have a particular part located within 1 μm of the linear waveguide 209, although is not limited thereto. The distance between the particular parts of the circular waveguide 208 and the linear waveguide 209 may be determined depending on various factors in the design stage. For example, the distance between the particular parts of the circular waveguide 208 and the linear waveguide 209 may be designed to have resonance occur at a particular frequency, based on a length of the cross section of the circular waveguide 208, a diameter of the linear waveguide 209, refractive indexes of the core 203 and the cladding 202, etc.

A process of receiving an echo ultrasound signal and calculating an acoustic pressure of the echo ultrasound signal will now be described in detail.

Upon reception of echo ultrasound signals reflected from the subject (ob), the optical resonating waveguide 220 may be forced by an acoustic pressure of the echo ultrasound signal. At least one of the linear waveguide 209 and the circular waveguide 208 forced by the acoustic pressure of the echo ultrasound signal may undergo a change in refractive index, or may be physically deformed. Both the change and deformation may occur as well. According to an exemplary embodiment, physical deformation refers to a substance being deformed when pressure is applied to the substance. For example, size, density, refractive index, etc., of the core 203 and cladding 202 may be changed, although exemplary embodiments are not limited thereto.

Accordingly, due to the physical deformation and change in refractive index that occurs when the echo ultrasound signal is received, the resonance frequency of an optical signal that flows between the linear waveguide 209 and the circular waveguide 208, which are slightly spaced apart, may be changed. In other words, the wavelength of an optical signal traveling from the linear waveguide 209 to the circular waveguide 208 or from the circular waveguide 208 to the linear waveguide 209 may be changed by an acoustic pressure.

Since the frequency is inversely proportional to the wavelength, the wavelength of the optical signal being resonated and traveling into the circular waveguide 208 may be changed as well. Hereinafter, the optical signal that is resonated and travels from the linear waveguide 209 to the circular waveguide 208 due to at least one of physical deformation and a change in refractive index may correspond to an optical delay signal. The optical delay signal may travel into the linear waveguide 209 via the circular waveguide 208.

The calculator 230 may calculate an acoustic pressure of the echo ultrasound signal based on a change between a predetermined resonance frequency of the optical signal and a resonance frequency of the optical delay signal, e.g., based on a change in wavelength of the optical signal. The wavelength of the optical signal that is resonated and travels from the linear waveguide 209 to the circular wave guide 208 may be changed in proportion to the acoustic pressure of the echo ultrasound signal. That is, the greater the acoustic pressure of the echo ultrasound signal, the larger the change between the predetermined resonance frequency of the optical signal and the resonance frequency of the optical delay signal. Accordingly, the calculator 230 may calculate an acoustic pressure of the echo ultrasound signal based on an extent of a change in wavelength of the optical signal.

The converter 240 may convert the echo ultrasound signal to an electric signal based on the acoustic pressure calculated based on the change in wavelength between the optical signal and the optical delay signal output at the output end 212. Accordingly, in the ultrasonic probe 200 in accordance with an exemplary embodiment, the piezoelectric membrane 204 is not used to receive the echo ultrasound signal, but the optical resonating waveguide 220 may act as a receiver.

As discussed above, a change in wavelength between the optical signal traveling from the linear waveguide 209 to the circular waveguide 208 and the optical delay signal and a level of an acoustic pressure of the echo ultrasound signal may be proportional to each other. The level of the acoustic pressure of the echo ultrasound signal may also be proportional to an extent of a change in intensity between the optical signal and the optical delay signal. Accordingly, the converter 240 may convert the echo ultrasound signal to an electric signal and output the electric signal, by determining the magnitude of the electric signal using the acoustic pressure of the echo ultrasound signal and the extent of a change in intensity between the optical signal and the optical delay signal. A configuration for conversion into and out of an electric signal will be described in detail later.

The controller 250 may control general operations of the ultrasonic probe 200. The controller 250 may be operated by a processor embedded in the ultrasonic probe 200, and may generate control signals to control the ultrasonic probe 200 and control respective operations of the aforementioned components.

For example, the controller 250 may be implemented as an Application Specific Integrated Circuit (ASIC) embedded in the ultrasonic probe 200. The ASIC may include control logic to control the transducers, thus controlling overall operations of the ultrasonic probe 200. The ASIC may also include control logic to control to what extent the electric signal is to be output, based on the acoustic pressure. Accordingly, the controller 250 may control conversion into an electric signal by controlling the converter 240. In addition, the controller 250 may generate a control signal to control the optical resonating waveguide 220, and accordingly, control the optical signal flowing in the optical resonating waveguide 220.

The electric signal converted by the converter 240 may be delivered to a signal processor 310 of the main unit 300 over a wired or wireless communication network. Although not shown, the ultrasonic probe 200 may deliver an analog signal that underwent beamforming through an analog beamformer. Furthermore, the ultrasonic probe 200 may deliver a digital signal that underwent beamforming through a digital beamformer and digital conversion. However, the type of signals to be delivered by the ultrasonic probe 200 is not limited thereto.

As such, the ultrasonic probe 200 in accordance with exemplary embodiments may overcome limitations in the related art of not improving both transmission sensitivity and reception sensitivity at the same time because there is a tradeoff between the transmission sensitivity and the reception sensitivity, by separating the transmitter 210 and the optical resonating waveguide 220 that acts as a receiver.

Moreover, the ultrasonic probe 200 may be able to suppress occurrence of electrical noise and operate at a lower voltage than the related art ultrasonic probes, by configuring the optical resonating waveguide 220 to receive the echo ultrasound signal.

The main unit 300 may be connected to the ultrasonic probe 200 through a wired or wireless communication network for controlling the ultrasonic probe 200 or may contain components to generate an ultrasound image based on the signal received from the ultrasonic probe 200.

Turning back to FIG. 4, the main unit 300 may include the signal processor 310, an image processor 320, and a system controller 330. The signal processor 310, the image processor 320, and the system controller 330 may be integrated in an SoC embedded in the main unit 300. In this regard, there may be multiple SoCs embedded in the main unit 300, and the aforementioned components are not limited to only being integrated in a single SoC.

The signal processor 310, the image processor 320, and the system controller 330 will now be described in more detail.

The signal processor 310 may convert a digital or analog signal received from the ultrasonic probe 200 into a format suitable for image processing. In a case in which an analog signal is received, the signal processor 310 may perform sampling on the analog signal with an Analog-to-Digital Converter (ADC), thus converting the analog signal into a digital form.

The signal processor 310 may perform filtering to eliminate noise outside a desired frequency band. For example, the signal processor 310 may perform low-pass filtering to eliminate noise of high frequency components with a Low-Pass Filter (LPF). As another example, the signal processor 310 may perform low-pass filtering with an anti-aliasing LPF to avoid aliasing due to high frequency components. However, noise filtering according to exemplary embodiments is not limited thereto.

The signal processor 310 may further include a Digital Signal Processor (DSP). The signal processor 310 may generate ultrasound image data by performing an envelope detection process on the digital signal to detect an amplitude of the echo ultrasound signal. Processes to be performed by the signal processor 310 are not limited to the above described examples, and various well-known processes may be used to generate ultrasound image data.

The image processor 320 may create an ultrasound image of a particular part of the subject (ob) to be watched by the user, e.g., a doctor or patient, based on the ultrasound image data. In addition, the image processor 230 may further perform separate additional image processing on the ultrasound image according to some exemplary embodiments. For example, the image processor 230 may further perform post image processing, such as compensating for or readjusting contrast, brightness, or sharpness of the ultrasound image. Such image processing of the image processor 320 may be performed according to a predetermined specification, or in response to a user command input through the input unit 350.

The system controller 330 may control general operations of the ultrasound imaging apparatus 10. For example, the system controller 330 may control respective operations of the signal processor 310, the image processor 320, and the display 340.

According to an exemplary embodiment, the system controller 330 may control operations of the ultrasonic imaging apparatus 10 according to a predetermined specification, or generate a predetermined control instruction in response to a user command input through the input unit 350 to control operations of the ultrasonic imaging apparatus 10.

The system controller 330 may include not only a processor, but also a Read Only Memory (ROM) for storing a control program for controlling the ultrasonic imaging apparatus 10, and a Random Access Memory (RAM) for storing signals input from the ultrasonic probe 200 or input unit 350, or ultrasound image data, or for being used as a memory space for various tasks performed by the ultrasonic imaging apparatus 10.

Alternatively, the processor, the RAM, and the ROM may be incorporated on a graphic processing board (not shown) physically separated from but electrically connected to the system controller 330. The processor, ROM, and RAM may be connected to one another via an internal bus. According to an exemplary embodiment, the term ‘system controller’ may refer to a component that includes a processor, RAM and ROM. The term ‘system controller’ may also refer to a component that includes a processor, a RAM, a ROM, and a processing board.

The display 340 may display the ultrasound image generated by the image processor 320, thereby enabling the user to visually examine the internal structure or tissue of the subject (ob).

The input unit 350 may receive a control command from the user to control the ultrasonic imaging apparatus 10. As discussed above, the input unit 350 may include, but is not limited to including, a user interface, such as a keyboard, a mouse, a trackball, a touch screen, a pedal, etc. An ultrasonic probe in a piezoelectric ultrasonic transducer type will now be described in more detail.

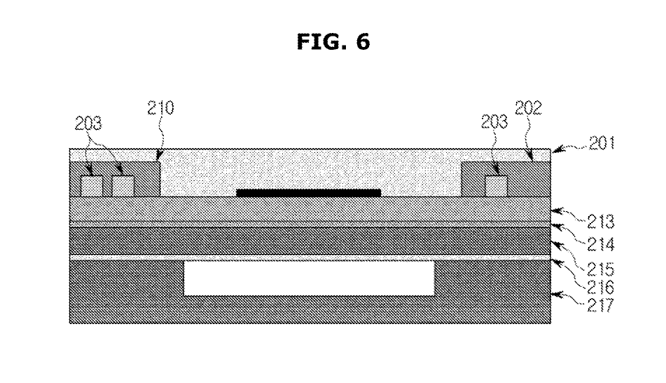

FIG. 6 illustrates an internal structure of an ultrasonic probe in a piezoelectric ultrasonic transducer type, according to an exemplary embodiment.

Referring to FIG. 6, an upper electrode 214 may be formed on the top of the piezoelectric layer 215 and a lower electrode 216 may be formed on the bottom of the piezoelectric layer 215. The piezoelectric layer 215 may include a piezoelectric substance, which performs conversion between electric and ultrasound signals while vibrating. According to an exemplary embodiment, when a voltage is applied across the upper electrode 214 and the lower electrode 216, an electric field is generated, inducing piezoelectric effect on the piezoelectric substance, which in turn vibrates. Accordingly, the piezoelectric substance may generate an ultrasound signal.

Since impedance of the piezoelectric layer 215 may be very high, a matching layer 213 may be included in the ultrasonic probe. The matching layer 213 may act to decrease a difference in impedance between the piezoelectric layer 215 and the subject, to deliver the ultrasound signal to the subject to the greatest extent possible. The ultrasound signal that passed through the matching layer 213 may be focused through a lens 201, to be transmitted to a particular part of the subject.

A sound-absorbing layer 217 may be arranged in a lower part of the ultrasonic probe. The sound-absorbing layer 217 may act to prevent distortion of the ultrasound image by blocking an ultrasound signal from proceeding in the opposite direction.

Referring to FIG. 6, optical resonating waveguides 220 may be arranged on the left and right sides of the ultrasonic probe. Details of the optical resonating waveguide 220 have already been described above, so the description will be omitted herein.

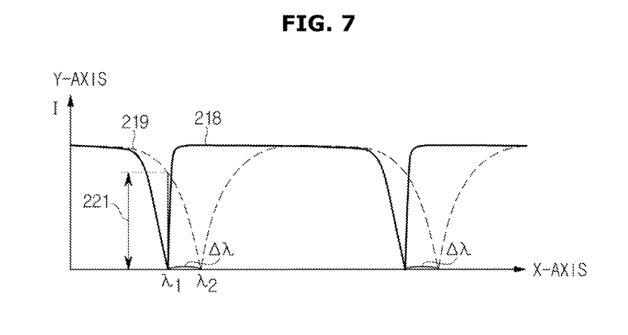

FIG. 7 shows a spectrum from which a difference in wavelength of an optical signal and an optical delay signal may be seen.

The X-axis of the spectrum corresponds to the wavelength λ of a signal, and the Y-axis corresponds to intensity I of the signal. An optical signal 218 flowing in the linear waveguide may be resonated and travel into the circular waveguide 208. As shown in FIG. 7, the wavelength λ1 of the optical signal being resonated and traveling into the circular waveguide 208 from the linear waveguide 209 may be pre-determined by the developer in designing the optical resonating waveguide.

Physical deformation and a change in refractive index of the core or cladding within the optical resonating waveguide may occur according to an acoustic pressure of the echo ultrasound signal. Specifically, there may be a difference between the wavelength λ2 of an optical delay signal 219 being resonated and traveling into the circular waveguide 208, and the wavelength λ1 of the optical signal 218 being resonated and traveling into the circular waveguide 208 before reception of the echo ultrasonic signal. Such a difference in wavelength between the optical signal 218 and the optical delay signal 219 may correspond to λ2-λ1, i.e., Δλ, as shown in FIG. 7.

The difference in wavelength between the optical signal and the optical delay signal being resonated and coming into the circular waveguide, Δλ, may be proportional to the acoustic pressure of the echo ultrasound signal. That is, the greater the acoustic pressure of the echo ultrasound signal, the larger the difference in wavelength Δλ.

Accordingly, the ultrasonic probe in accordance with an exemplary embodiment may calculate an acoustic pressure of the echo ultrasound signal not using the piezoelectric substance but based on a change in wavelength detected using the optical resonating waveguide.

The ultrasonic probe may output an electric signal based on the calculated acoustic pressure. According to an exemplary embodiment, the respective intensity of the optical signal and optical delay signal may be different in a section where the wavelength of the optical signal traveling into the circular waveguide 208 from the linear waveguide 209 is changed. As shown in FIG. 7, comparing intensities of the optical signal 218 and optical delay signal 219 at a same X coordinate value, i.e., at a same wavelength in a section between λ1 and λ2, it may be seen that there is a difference in intensity between the optical signal 218 and the optical delay signal 219. The ultrasonic probe may determine the magnitude of the electric signal depending on how much the intensity of the optical signal is changed.

According to an exemplary embodiment, the ultrasonic probe may determine the magnitude of the electric signal using an acoustic pressure of the echo ultrasound signal, which is calculated based on a change in intensity 221 and a change in wavelength Δλ between the optical signal 218 and the optical delay signal 219 at a wavelength, λ1. The change in wavelength Δλ between the optical delay signal 219 and the optical signal 218 traveling into the circular waveguide from the linear waveguide may be proportional to the level of the acoustic pressure of the echo ultrasound signal. Furthermore, the level of the acoustic pressure of the echo ultrasound signal may be proportional to an extent of a change in intensity 221 between the optical signal 218 and the optical delay signal 219. Accordingly, the ultrasonic probe may convert the echo ultrasonic signal to an electric signal and output the electric signal, by determining to what extent the magnitude of the electric signal is to be adjusted by using the change in intensity 221 between the optical signal 218 and the optical delay signal 219 and the level of the acoustic pressure.

In the ASIC embedded in the ultrasonic probe in accordance with an exemplary embodiment, there may be predetermined control logic to control the magnitude of an electric signal to be output, depending on the level of the acoustic pressure, e.g., an extent of the change in wavelength. Accordingly, the ASIC may control the magnitude of the electric signal to be output based on the level of the acoustic pressure. With the method, the ultrasonic prove may convert the echo ultrasound signal to an electric signal and output the electric signal, in a faster way.

The ultrasonic probe in accordance with an exemplary embodiment may overcome shortcomings in the related art that it is difficult to increase transmission sensitivity and reception sensitivity at the same time, by separating configurations that perform transmission and reception. Structures of the core and cladding included in the linear and circular waveguides will now be examined.

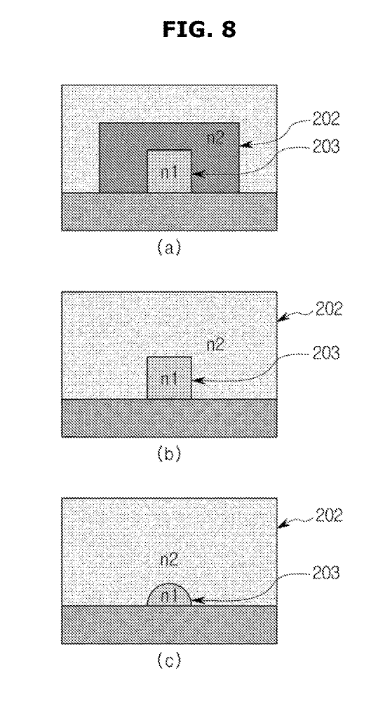

FIG. 8 illustrates structures of a core and a cladding, according to an exemplary embodiment.

A core and a cladding are substances that make up a waveguide used for the purpose of transmission of optical signals, and may be implemented with optical fiber capable of transmitting optical signals. As discussed above, the core should be implemented with a refractive index greater than a refractive index of the cladding, and the core and the cladding may be implemented as any well-known substance, such as polymer that may form the optical fiber, although are not limited thereto. Referring to FIG. 8, assuming that refractive indexes of the core 203 and cladding 202 are n1 and n2, respectively, a relationship between n1 and n2 is to be n1>n2.

As shown in (a) of FIG. 8, the core 203 is surrounded by the cladding 202. The core 203 implemented by a substance with a greater refractive index than the cladding 202 may suppress, to the greatest extent possible, leakage of an optical signal flowing in the core 203 out to the cladding 202.

Alternatively, referring to (b) of FIG. 8, the ultrasonic probe in accordance with an exemplary embodiment may not separately include the cladding 202, but may have a lens that is arranged in an upper part act as the cladding. In other words, the ultrasonic probe 200 may use the lens to perform the functionality of the cladding 202 that prevents leakage of an optical signal as well as the functionality to focus the ultrasound signal to be irradiated to a particular spot. The refractive index of the core 203 should be greater than that of the lens, and the lens may be implemented with the same substance as the cladding 202.

As shown in (a) of FIG. 8, the core and cladding may be implemented in a rectangular shape. Alternatively, as shown in (c) of FIG. 8, the core may be implemented in a circular shape. Also, the cladding may be implemented in a circular shape as well. However, the shape of the core or cladding is not limited to a particular shape.

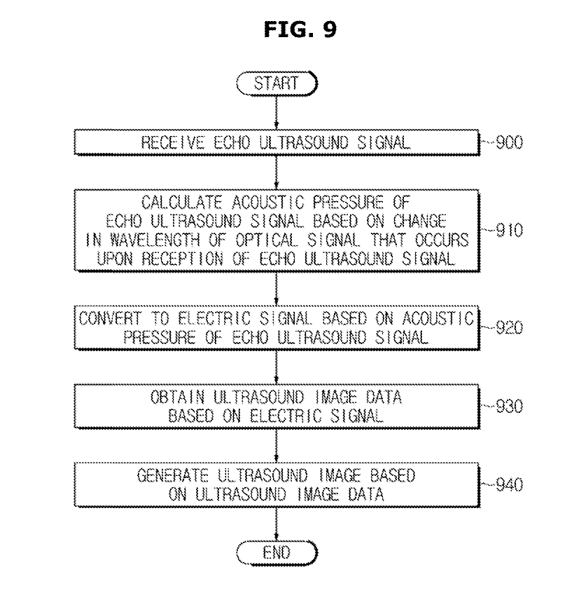

FIG. 9 is a flowchart illustrating a method for controlling an ultrasonic imaging apparatus, according to an exemplary embodiment.

The ultrasonic imaging apparatus may use the optical resonating waveguide of the ultrasonic probe to receive an echo ultrasound signal, in operation 900. The optical resonating waveguide may be changed in characteristics or physically deformed, according to an acoustic pressure of the ultrasound signal. Specifically, at least one of a change in an internal refractive index and physical deformation may occur in the optical resonating waveguide according to the acoustic pressure of the ultrasound signal. Accordingly, the resonance frequency at which resonance occurs when the optical signal travels between the linear waveguide and the circular waveguide, or the wavelength that corresponds to the resonance frequency, may be changed.

Therefore, upon reception of the echo ultrasound signal, the ultrasonic imaging apparatus may determine a change in frequency at which the optical signal is resonated within the optical resonating waveguide, e.g., a change in wavelength of the optical signal, and based on the change, calculate an acoustic pressure of the echo ultrasound signal, in operation 910. The change in wavelength of the optical signal may be proportional to the acoustic pressure of the echo ultrasound signal.

The ultrasonic imaging apparatus may calculate the acoustic pressure of the echo ultrasound signal using the optical signal flowing in the optical resonating waveguide, thereby minimizing electrical noise that occurs in calculating the acoustic pressure, and thus obtaining a more accurate acoustic pressure of the echo ultrasound signal.

The ultrasonic imaging apparatus may include control logic to control input and output ends of the linear waveguide. The control logic may be integrated in an ASIC embedded in the ultrasonic imaging apparatus. The control logic may control the flow of an optical signal that flows in the linear waveguide. Furthermore, the control logic may determine a change in wavelength of the optical signal flowing between the input and output ends, and based on the change in wavelength, calculate an acoustic pressure of the echo ultrasound signal.

The ultrasonic imaging apparatus may convert the echo ultrasound signal to an electric signal based on the acoustic pressure, in operation 920. The ultrasonic imaging apparatus may determine a change in intensity of the optical signal in a section where the wavelength of the optical signal being resonated and traveling from the linear waveguide to the circular waveguide is changed. Accordingly, the ultrasonic imaging apparatus may output the electric signal using an extent of the change in intensity of the optical signal and the level of the acoustic pressure. The magnitude of the output electric signal may be proportional to the extent of the change in intensity of the optical signal and the level of the acoustic pressure.

The ultrasonic imaging apparatus may obtain ultrasound image data based on the output electric signal in operation 930. According to an exemplary embodiment, the term ultrasonic image data as used herein refers to data obtained by converting the electric signal corresponding to an analog or digital signal into a format suitable for image processing. For example, the ultrasonic imaging apparatus may obtain a digital signal by performing beamforming on the electric signal and sampling the digital signal through an ADC.

Furthermore, the ultrasonic imaging apparatus may use an LPF to eliminate noise of the digital signal outside the desired frequency band. The ultrasonic imaging apparatus may also perform an envelope detection process on the digital signal that underwent the LPF to detect an amplitude of the echo ultrasound signal, thereby obtaining ultrasonic image data, in operation 930. Furthermore, the ultrasonic imaging apparatus may include various components used to obtain the ultrasound image data, such as a decimator, a gain control module, a mixer, etc.

The ultrasonic imaging apparatus may generate an ultrasound image of an internal part of the subject based on the ultrasound image data, in operation 940. For example, the ultrasonic imaging apparatus may include a Digital Scan Converter (DSC) for performing scan conversion. The ultrasonic imaging apparatus may use the DSC to scan-convert the ultrasonic image data, thereby generating an ultrasound image, such as an A-mode image, a B-mode image, and an M-mode image. In addition, the ultrasonic imaging apparatus may further include a DSP. The ultrasonic imaging apparatus may use the DSP to create a D-mode image and/or a C-mode image from the ultrasound image data.

The ultrasonic imaging apparatus may provide the ultrasound image for the user through an internal display. Moreover, the ultrasonic imaging apparatus may work with an external display over a wired or wireless communication network, and provide the ultrasound image for the user through the external display.

The method according to the exemplary embodiments may be implemented in program instructions which are executable by various computing devices and recorded in computer-readable media. The computer-readable media may include program instructions, data files, data structures, etc., separately or in combination. The program instructions recorded on the computer-readable media may be designed and configured specially for the exemplary embodiments, or may be well-known to people having ordinary skill in the art of computer software. Examples of the computer readable recording media include read-only memory (ROM), random-access memory (RAM), Compact Disc (CD)-ROMs, magnetic tapes, floppy disks, optical data storage devices, etc. The computer readable recording medium can also be distributed over network coupled computer systems so that the computer readable code is stored and executed in a distributed fashion.

Examples of the program instructions include not only machine language codes but also high-level language codes which are executable by various computing devices using an interpreter. The aforementioned hardware devices may be configured to operate as one or more software modules to carry out exemplary embodiments, and vice versa.

Although the exemplary embodiments are described with reference to certain exemplary embodiments as described above and the accompanying drawings, it will be apparent to those of ordinary skill in the art that various modifications and changes can be made to the exemplary embodiments. For example, the aforementioned method may be performed in a different order, and/or the aforementioned systems, structures, devices, circuits, etc., may be combined in different combinations from what is described above, and/or replaced or substituted by other components or equivalents thereof, to obtain appropriate results.

Therefore, other implementations, other exemplary embodiments, and equivalents thereof may fall within the following claims.

What is claimed is:

1. An ultrasonic probe comprising:
an optical resonating waveguide configured to receive an echo ultrasound signal;
a calculator configured to calculate an acoustic pressure of the echo ultrasound signal, based on a change in wavelength of an optical signal traveling within the optical resonating waveguide, the change occurring in response to the optical resonating waveguide receiving the echo ultrasound signal; and
a converter configured to convert the echo ultrasound signal to an electric signal based on the acoustic pressure of the echo ultrasound signal.

2. The ultrasonic probe of claim 1, wherein the optical resonating waveguide comprises a linear waveguide and a circular waveguide, and
wherein the calculator is configured to:
detect the change in the wavelength of the optical signal being resonated and traveling from the linear waveguide to the circular waveguide based on at least one of a change in refractive index and physical deformation of the optical resonating waveguide that occur in response to the optical resonating waveguide receiving the echo ultrasound signal.

3. The ultrasonic probe of claim 1, wherein the optical resonating waveguide comprises a linear waveguide and a circular waveguide, and the converter is configured to:
determine a change in intensity of the optical signal traveling from the linear waveguide to the circular waveguide based on the acoustic pressure of the echo ultrasound signal, and convert the echo ultrasound signal to the electric signal based on the acoustic pressure of the echo ultrasound signal and the change in the intensity of the optical signal.

4. The ultrasonic probe of claim 1,
further comprising a controller configured to control a flow of the optical signal within the optical resonating waveguide.

5. The ultrasonic probe of claim 1, further comprising:
at least one of a piezoelectric layer and a piezoelectric membrane; and
a transmitter configured to transmit an ultrasound signal converted from an electric signal based on at least one of the piezoelectric layer and the piezoelectric membrane to a subject.

6. The ultrasonic probe of claim 1,
wherein the optical resonating waveguide further comprises a linear waveguide and a circular waveguide, each of the linear waveguide and the circular waveguide comprising a core and a cladding, and
wherein the core and the cladding are implemented as at least one of a circular shape or a polygonal shape.

7. The ultrasonic probe of claim 6,
wherein the cladding is implemented as a lens configured to focus an ultrasound signal to be transmitted to a subject.

8. An ultrasonic imaging apparatus comprising:
an optical resonating waveguide configured to receive an echo ultrasound signal;
a converter configured to output an electric signal according to an acoustic pressure of the ultrasound signal;
a signal processor configured to obtain ultrasound image data based on the electric signal; and
an image processor configured to generate an ultrasound image based on the ultrasound image data.

9. The ultrasonic imaging apparatus of claim 8, wherein the optical resonating waveguide comprises a linear waveguide and a circular waveguide; and
wherein the ultrasonic imaging apparatus further comprises a calculator configured to detect a change in wavelength of an optical signal being resonated and traveling from the linear waveguide to the circular waveguide based on at least one of a change in refractive index and physical deformation of the optical resonating waveguide that occur in response to the optical resonating waveguide receiving the echo ultrasound signal, and calculate the acoustic pressure of the echo ultrasound signal based on the change in the wavelength.

10. The ultrasonic imaging apparatus of claim 8, wherein the optical resonating waveguide comprises a linear waveguide and a circular waveguide, and
wherein the converter is configured to determine a change in intensity of an optical signal traveling from the linear waveguide to the circular waveguide based on the acoustic pressure of the echo ultrasound signal, and output an electric signal converted based on the acoustic pressure of the echo ultrasound signal and the change in the intensity of the optical signal.

11. The ultrasonic imaging apparatus of claim 8, further comprising:
at least one of a piezoelectric layer and a piezoelectric membrane; and
a transmitter configured to transmit an ultrasound signal converted from an electric signal based on at least one of the piezoelectric layer and the piezoelectric membrane to a subject.

12. The ultrasonic imaging apparatus of claim 8,
wherein the optical resonating waveguide further comprises a linear waveguide and a circular waveguide, each of the linear waveguide and the circular waveguide comprising a core and a cladding, and
wherein the core and the cladding are implemented as at least one of a circular shape and a polygonal shape.

13. The ultrasonic imaging apparatus of claim 12,
wherein the cladding is implemented as a lens configured to focus an ultrasound signal to be transmitted to a subject.

14. A method for controlling an ultrasound imaging apparatus, the method comprising:
receiving, by an optical resonating waveguide, an echo ultrasound signal;
outputting an electric signal according to an acoustic pressure of the echo ultrasound signal;
obtaining ultrasound image data based on the electric signal; and
generating an ultrasound image based on the ultrasound image data.

15. The method of claim 14,
wherein the optical resonating waveguide comprises a linear waveguide and a circular waveguide, and
the outputting comprises:
detecting a change in a wavelength of an optical signal being resonated and traveling from the linear waveguide to the circular waveguide based on at least one of a change in refractive index and physical deformation of the optical resonating waveguide that occur upon the receiving of the echo ultrasound signal.

16. The method of claim 14,
wherein the optical resonating waveguide comprises a linear waveguide and a circular waveguide, and
the outputting comprises:
determining a change in intensity of an optical signal traveling from the linear waveguide to the circular waveguide based on the acoustic pressure of the echo ultrasound signal, and outputting the electric signal which is converted based on the acoustic pressure of the echo ultrasound signal and the change in the intensity of the optical signal.

17. The method of claim 14,
wherein the ultrasound imaging apparatus comprises at least one of a piezoelectric layer and a piezoelectric membrane, and
wherein the method further comprises: transmitting an ultrasound signal converted from an electric signal based on the at least one of the piezoelectric layer and the piezoelectric membrane to a subject.

18. The method of claim 14,
wherein the optical resonating waveguide comprises a linear waveguide and a circular waveguide, and each of the linear waveguide and the circular waveguide comprises a core and a cladding, and
wherein the core and the cladding are implemented as at least one of a circular shape and a polygonal shape.

19. The method of claim 14,
wherein the cladding is implemented as a lens configured to focus an ultrasound signal to be transmitted to a subject.

20. An ultrasonic probe, comprising:
an optical resonating waveguide configured to receive an ultrasound signal reflected from a subject, the optical resonating waveguide comprising a portion through which an optical signal is configured to travel; and
a controller configured to calculate acoustic pressure generated in response to the received ultrasound signal, based on a change in a resonance frequency of the optical signal.

21. The ultrasonic probe of claim 20, wherein the portion comprises a linear waveguide and a circular waveguide connected to a portion of the linear waveguide, each of the linear waveguide and the circular waveguide being implemented as a ring resonator type.

22. The ultrasonic probe of claim 20, wherein the optical resonating waveguide is configured to totally internally reflect the optical signal.
